SEQUENCE LISTING (8 sequences)

SEQ ID NO: 1 - DNA, 322 bp, synthetic construct
  source 1..322: mol_type = other DNA
ttagggttcaggcctgagcccctgctgctatccctccttcaaaggaggagatcaaggagcttagggtcccccgcacaggcccaccccagggtggggttcttcctttgaagggaattgctttggggtggggtcggttctatctgctccactctgtggctgacagtttctccaaggggctgcaggtgtcagctgtctgagcccgggctgagctctgaaacgtgcctactcaaacttcccgtggggtaggggaggcccagaaccaccctctgagagtggcaaaaagtggtcctggagccaggggaaatgtggatggggtagagat

SEQ ID NO: 2 - DNA, 116 bp, synthetic construct
  source 1..116: mol_type = other DNA
cttatcaaaattgagtcacttggacacatccagctgtgagggaggcctcaacttcctggaagttgggttttattttgcttgggcaagttggccatatgcccccaactgcctattta

SEQ ID NO: 3 - DNA, 190 bp, synthetic construct
  source 1..190: mol_type = other DNA
agcctgattctgcttaactttttcccttgactttggcattttcactttgacatgttccctgagagcctggggggtggggaacccagctccagctggtgacgtttggggccggcccaggcctagggtgtggaggagccttgccatcgggcttcctgtctctcttcatttaagcacgactctgcagaaggaa

SEQ ID NO: 4 - DNA, 79 bp, synthetic construct
  source 1..79: mol_type = other DNA
caaagcaccctccccactgggctcctggttgcagagctccaagtcctcacacagatacgcctgtttgagaagcagcggg

SEQ ID NO: 5 - DNA, 135 bp, synthetic construct
  source 1..135: mol_type = other DNA
caaagacgcaagcccagaggtaaggtggtcagactcggcttccttccccggagctgagagggaggggaacgtggggcagatgcacaggaagaatttctgtgctctgcccagttgtctgctttagataaatatgtc

SEQ ID NO: 6 - DNA, 187 bp, synthetic construct
  source 1..187: mol_type = other DNA
tgggggttatggggagaagataaaagtgcctgtgggaccacagactctcgctgtggtggagctgggccctcttaccctcccaagcctcgcccctcatcccatccctgggggccaggggtgagggcggcaggaacctcaaggctctgagaaagtgcgtggtgtgtgttgccattttggtctcttctcc

SEQ ID NO: 7 - DNA, 1029 bp, synthetic construct
  source 1..1029: mol_type = other DNA
ttagggttcaggcctgagcccctgctgctatccctccttcaaaggaggagatcaaggagcttagggtcccccgcacaggcccaccccagggtggggttcttcctttgaagggaattgctttggggtggggtcggttctatctgctccactctgtggctgacagtttctccaaggggctgcaggtgtcagctgtctgagcccgggctgagctctgaaacgtgcctactcaaacttcccgtggggtaggggaggcccagaaccaccctctgagagtggcaaaaagtggtcctggagccaggggaaatgtggatggggtagagatcttatcaaaattgagtcacttggacacatccagctgtgagggaggcctcaacttcctggaagttgggttttattttgcttgggcaagttggccatatgcccccaactgcctatttaagcctgattctgcttaactttttcccttgactttggcattttcactttgacatgttccctgagagcctggggggtggggaacccagctccagctggtgacgtttggggccggcccaggcctagggtgtggaggagccttgccatcgggcttcctgtctctcttcatttaagcacgactctgcagaaggaacaaagcaccctccccactgggctcctggttgcagagctccaagtcctcacacagatacgcctgtttgagaagcagcgggcaaagacgcaagcccagaggtaaggtggtcagactcggcttccttccccggagctgagagggaggggaacgtggggcagatgcacaggaagaatttctgtgctctgcccagttgtctgctttagataaatatgtctgggggttatggggagaagataaaagtgcctgtgggaccacagactctcgctgtggtggagctgggccctcttaccctcccaagcctcgcccctcatcccatccctgggggccaggggtgagggcggcaggaacctcaaggctctgagaaagtgcgtggtgtgtgttgccattttggtctcttctcc

SEQ ID NO: 8 - DNA, 1085 bp, synthetic construct
  source 1..1085: mol_type = other DNA
ttgaattcggcatgcttatcgattttagggttcaggcctgagcccctgctgctatccctccttcaaaggaggagatcaaggagcttagggtcccccgcacaggcccaccccagggtggggttcttcctttgaagggaattgctttggggtggggtcggttctatctgctccactctgtggctgacagtttctccaaggggctgcaggtgtcagctgtctgagcccgggctgagctctgaaacgtgcctactcaaacttcccgtggggtaggggaggcccagaaccaccctctgagagtggcaaaaagtggtcctggagccaggggaaatgtggatggggtagagatcttatcaaaattgagtcacttggacacatccagctgtgagggaggcctcaacttcctggaagttgggttttattttgcttgggcaagttggccatatgcccccaactgcctatttaagcctgattctgcttaactttttcccttgactttggcattttcactttgacatgttccctgagagcctggggggtggggaacccagctccagctggtgacgtttggggccggcccaggcctagggtgtggaggagccttgccatcgggcttcctgtctctcttcatttaagcacgactctgcagaaggaacaaagcaccctccccactgggctcctggttgcagagctccaagtcctcacacagatacgcctgtttgagaagcagcgggcaaagacgcaagcccagaggtaaggtggtcagactcggcttccttccccggagctgagagggaggggaacgtggggcagatgcacaggaagaatttctgtgctctgcccagttgtctgctttagataaatatgtctgggggttatggggagaagataaaagtgcctgtgggaccacagactctcgctgtggtggagctgggccctcttaccctcccaagcctcgcccctcatcccatccctgggggccaggggtgagggcggcaggaacctcaaggctctgagaaagtgcgtggtgtgtgttgccattttggtctcttctccggcgcgcccgggatccacgcgtctgaattcga

(19) United States
(12) Patent Application Publication
(10) Pub. No.: US 2026/0297619 A1
(43) Pub. Date: Oct. 1, 2026

(54) Polynuceotide For T Cell Specific Transgene Expression

(71) Applicants: FUNDACION PUBLICA ANDALUZA PROGRESO Y SALUD M.P.- FPS, Sevilla (ES); Lentistem Biotech S.L., Granada (ES); Universidad de Granada, Granada (ES)

(72) Inventors: Francisco MARTIN MOLINA, Granada (ES); Noelia MALDONADO PÉREZ, Sevilla (ES); Maria TRISTAN MANZANO, Granada (ES); Pedro Luis JUSTICIA LIRIO, Granada (ES)

(21) Appl. No.: 19/479,143

(22) PCT Filed: Apr. 24, 2024
(86) PCT No.: PCT/EP2024/061307

§ 371 Date: Oct. 27, 2025

(30) Foreign Application Priority Data
Apr. 27, 2023 (EP) 23382393.9

(51) Int. Cl. C12N 15/86; A61K 40/11; A61K 40/31; C07K 16/00; C12N 5/0783

(52) U.S. Cl. CPC C12N 15/86; A61K 40/11; A61K 40/31; C07K 16/00; C12N 5/0636; C12N 2510/00; C12N 2740/15043

(57) ABSTRACT

The present invention refers to the medical field. Particularly, the present invention refers to a polynucleotide for T cell specific transgene expression, as well as to a method to generate CAR-T cells.

### FIELD OF THE INVENTION

The present invention refers to the medical field. Particularly, the present invention refers to a polynucleotide for T cell specific transgene expression, as well as to a method to generate CAR-T cells by using said polynucleotide.

### STATE OF THE ART

T lymphocytes constitute the basis of multiple cellular therapies due to their easy isolation and manipulation and their functional and migration properties, which make them excellent candidates for immunotherapy. With the new advanced technologies, genetic engineering of T lymphocytes can be easily addressed opening an endless of possibilities of therapeutic applications. Genetically engineered T cell immunotherapies have provided impressive efficacy treating B cell malignancies, endowing T cells with a chimeric antigen receptor to specifically recognize the antigen. These approaches can be applied to multiple diseases, from other cancers to infectious and autoimmune problems.

Chimeric antigen receptor (CAR) T cells (CAR-T cells) have become one of the most promising approaches for the treatment of cancer. CAR-T cell therapy represents a major advancement in personalized cancer treatment. In this strategy, patient's own T cells are genetically engineered to express a synthetic receptor that binds a tumor antigen. CAR T cells are then expanded for clinical use and infused back into the patient's body to attack and destroy chemotherapy-resistant cancer. Traditionally, the CAR is transduced into T cells via viral vectors, such as lentiviruses (LVs). Lentiviral particles are packaged in producer cell lines such as HEK293T cells. Upon co-transfection of different plasmids, one of which contains the CAR transgene, all required sequences are available to produce and package a viral particle containing the transgene of interest. Some lentiviral vectors, such as those derived from HIV-1 have, already been approved by the FDA/EMA.

Dramatic clinical responses and high rates of complete remission have been observed in the setting of CAR T-cell therapy of B-cell malignancies. This resulted in recent FDA/EMA approvals of CAR T cells directed against the CD19 protein (αCD19-CAR-T cells) for treatment of acute lymphoblastic leukemia and diffuse large B-cell lymphoma. These advanced therapy medicinal products (ATMPs) include: tisagenlecleucel (Kymriah, Novartis), axicabtageneciloleucel (Yescarta, Kite-Gilead), brexucabtagene autoleucel (Tecartus, Kite-Gilead) and lisocabtagenemaraleucel (Breyanzi, Bristol Myers Squibb). Recently idecabtagene vicleucel (Abecma, Bristol Myers Squibb) and ciltacabtagene autoleucel (Carvykti, Janssen) were also approved as αBCMA-CAR-T ATMPs for the treatment of refractory Multiple Myeloma. However, despite the impressive results of αCD19-CAR-T cells, there are still several aspects that must be improved. Aggressive severe side effects due to CAR-T overstimulation, such as cytokine release syndrome and neuroinflammation are common among treated patients and can lead to fatal outcomes. Furthermore, sustained complete remissions range from 42% to 62% for patients treated with commercial ATMPs, evidencing much room for improvement.

Different works have uncovered the importance of controlling CAR expression levels at the surface of the CAR-T cells to optimize its therapeutic activity. Despite this, all four approved αCD19-CAR-T cells and most of those that are being tested in ongoing clinical trials use autologous T cells transduced with retroviral vectors expressing a αCD19-CAR through constitutive, strong promoters such as the human EF1α and the murine stem cell virus LTR (MSCV). High CARs concentrations on the T cell surface can result in spontaneous clustering of the CARs (independent of the ligand) leading to tonic-signalling. This tonic signalling can affect safety (pro-inflammatory cytokines secretion in non-target tissues) and efficacy (premature exhaustion due to continuous proliferation) of CAR-T cells. Furthermore, high-density and constitutive CAR presence on the T cell surface can also lead to overstimulation upon antigen recognition, which also affects safety (excess of pro-inflammatory cytokine secretion) and efficacy (early exhaustion, apoptosis and loss of naive/stem cell memory T cells phenotype) of the CAR-T cells.

So, there is an unmet medical need to improve the efficacy and safety of CAR-T cells or fourth generation CAR-T cells or TRUCKs (T cells redirected for antigen-unrestricted cytokine-initiated killing'). In particular, there is an unmet medical need for the development of strategies aimed at generating CAR-T cells that express the CAR in a more physiological manner than that provided by strong promoters. Furthermore, tissue-restricted gene expression can be useful in improving the manufacturing of CAR-T or TRUCK cells, as it would allow for the production of LVs without expressing the transgene at the packaging cells which, in case of toxicity, can significantly reduce viral titer. The present invention is focused on solving this problem, and a polynucleotide sequence for T cell specific transgene expression is herein provided.

### DESCRIPTION OF THE INVENTION

### Brief Description of the Invention

The present invention refers to a polynucleotide sequence for T cell specific transgene expression, as well as to a method to generate CAR-T cells by using said polynucleotide.

The inventors of the present invention identified the necessity to generate CAR-T cells that express the CAR in a more physiological manner than that provided by strong promoters. They hypothesized that restricting transgene expression to T cells, using a T-cell specific promoter, could solve some of the safety concerns associated to CAR-T cell therapies. For this purpose, the inventors generated a synthetic polynucleotide comprising or consisting of SEQ ID NO: 7 that is based on regulatory regions of the human CD4 locus (FIG. 1). As shown in FIG. 1, SEQ ID NO: 7 comprises or consists of the sequences: SEQ ID NO: 1, SEQ ID NO: 2, SEQ ID NO: 3, SEQ ID NO: 4, SEQ ID NO: 5 and SEQ ID NO: 6. SEQ ID NO: 8 comprises or consists of SEQ ID NO:7 and accessory sequences containing restriction sites.

The sequences included in the present application are summarised below:

SEQ ID NO: 1:
TTAGGGTTCAGGCCTGAGCCCCTGCTGCTATCCCTCCTTCAAAGGAGGA
GATCAAGGAGCTTAGGGTCCCCCGCACAGGCCCACCCCAGGGTGGGGTT
CTTCCTTTGAAGGGAATTGCTTTGGGGTGGGGTCGGTTCTATCTGCTCC
ACTCTGTGGCTGACAGTTTCTCCAAGGGGCTGCAGGTGTCAGCTGTCTG
AGCCCGGGCTGAGCTCTGAAACGTGCCTACTCAAACTTCCCGTGGGGTA
GGGGAGGCCCAGAACCACCCTCTGAGAGTGGCAAAAAGTGGTCCTGGAG
CCAGGGGAAATGTGGATGGGGTAGAGAT
SEQ ID NO: 2:
CTTATCAAAATTGAGTCACTTGGACACATCCAGCTGTGAGGGAGGCCTC
AACTTCCTGGAAGTTGGGTTTTATTTTGCTTGGGCAAGTTGGCCATATG
CCCCCAACTGCCTATTTA
SEQ ID NO: 3:
AGCCTGATTCTGCTTAACTTTTTCCCTTGACTTTGGCATTTTCACTTTG
ACATGTTCCCTGAGAGCCTGGGGGGTGGGGAACCCAGCTCCAGCTGGTG
ACGTTTGGGGCCGGCCCAGGCCTAGGGTGTGGAGGAGCCTTGCCATCGG
GCTTCCTGTCTCTCTTCATTTAAGCACGACTCTGCAGAAGGAA
SEQ ID NO: 4:
CAAAGCACCCTCCCCACTGGGCTCCTGGTTGCAGAGCTCCAAGTCCTCA
CACAGATACGCCTGTTTGAGAAGCAGCGGG
SEQ ID NO: 5:
CAAAGACGCAAGCCCAGAGGTAAGGTGGTCAGACTCGGCTTCCTTCCCC
GGAGCTGAGAGGGAGGGGAACGTGGGGCAGATGCACAGGAAGAATTTCT
GTGCTCTGCCCAGTTGTCTGCTTTAGATAAATATGTC
SEQ ID NO: 6:
TGGGGGTTATGGGGAGAAGATAAAAGTGCCTGTGGGACCACAGACTCTC
GCTGTGGTGGAGCTGGGCCCTCTTACCCTCCCAAGCCTCGCCCCTCATC
CCATCCCTGGGGGCCAGGGGTGAGGGCGGCAGGAACCTCAAGGCTCTGA
GAAAGTGCGTGGTGTGTGTTGCCATTTTGGTCTCTTCTCC
SEQ ID NO: 7:
TTAGGGTTCAGGCCTGAGCCCCTGCTGCTATCCCTCCTTCAAAGGAGGA
GATCAAGGAGCTTAGGGTCCCCCGCACAGGCCCACCCCAGGGTGGGGTT
CTTCCTTTGAAGGGAATTGCTTTGGGGTGGGGTCGGTTCTATCTGCTCC
ACTCTGTGGCTGACAGTTTCTCCAAGGGGCTGCAGGTGTCAGCTGTCTG
AGCCCGGGCTGAGCTCTGAAACGTGCCTACTCAAACTTCCCGTGGGGTA
GGGGAGGCCCAGAACCACCCTCTGAGAGTGGCAAAAAGTGGTCCTGGAG
CCAGGGGAAATGTGGATGGGGTAGAGATCTTATCAAAATTGAGTCACTT
GGACACATCCAGCTGTGAGGGAGGCCTCAACTTCCTGGAAGTTGGGTTT
TATTTTGCTTGGGCAAGTTGGCCATATGCCCCCAACTGCCTATTTAAGC
CTGATTCTGCTTAACTTTTTCCCTTGACTTTGGCATTTTCACTTTGACA
TGTTCCCTGAGAGCCTGGGGGGTGGGGAACCCAGCTCCAGCTGGTGACG
TTTGGGGCCGGCCCAGGCCTAGGGTGTGGAGGAGCCTTGCCATCGGGCT
TCCTGTCTCTCTTCATTTAAGCACGACTCTGCAGAAGGAACAAAGCACC
CTCCCCACTGGGCTCCTGGTTGCAGAGCTCCAAGTCCTCACACAGATAC
GCCTGTTTGAGAAGCAGCGGGCAAAGACGCAAGCCCAGAGGTAAGGTGG
TCAGACTCGGCTTCCTTCCCCGGAGCTGAGAGGGAGGGGAACGTGGGGC
AGATGCACAGGAAGAATTTCTGTGCTCTGCCCAGTTGTCTGCTTTAGAT
AAATATGTCTGGGGGTTATGGGGAGAAGATAAAAGTGCCTGTGGGACCA
CAGACTCTCGCTGTGGTGGAGCTGGGCCCTCTTACCCTCCCAAGCCTCG
CCCCTCATCCCATCCCTGGGGGCCAGGGGTGAGGGCGGCAGGAACCTCA
AGGCTCTGAGAAAGTGCGTGGTGTGTGTTGCCATTTTGGTCTCTTCTCC
SEQ ID NO: 8:
TTGAATTCGGCATGCTTATCGATTTTAGGGTTCAGGCCTGAGCCCCTGC
TGCTATCCCTCCTTCAAAGGAGGAGATCAAGGAGCTTAGGGTCCCCCGC
ACAGGCCCACCCCAGGGTGGGGTTCTTCCTTTGAAGGGAATTGCTTTGG
GGTGGGGTCGGTTCTATCTGCTCCACTCTGTGGCTGACAGTTTCTCCAA
AGCCCGGGCTGAGCTCTGAAACGTGCCTACTCAAACTTCCCGTGGGGTA
GGGGGCTGCAGGTGTCAGCTGTCTGGGGAGGCCCAGAACCACCCTCTGA
GAGTGGCAAAAAGTGGTCCTGGAGCCAGGGGAAATGTGGATGGGGTAGA
GATCTTATCAAAATTGAGTCACTTGGACACATCCAGCTGTGAGGGAGGC
CTCAACTTCCTGGAAGTTGGGTTTTATTTTGCTTGGGCAAGTTGGCCAT
ATGCCCCCAACTGCCTATTTAAGCCTGATTCTGCTTAACTTTTTCCCTT
GACTTTGGCATTTTCACTTTGACATGTTCCCTGAGAGCCTGGGGGGTGG
GGAACCCAGCTCCAGCTGGTGACGTTTGGGGCCGGCCCAGGCCTAGGGT
GTGGAGGAGCCTTGCCATCGGGCTTCCTGTCTCTCTTCATTTAAGCACG
ACTCTGCAGAAGGAACAAAGCACCCTCCCCACTGGGCTCCTGGTTGCAG
AGCTCCAAGTCCTCACACAGATACGCCTGTTTGAGAAGCAGCGGGCAAA
GACGCAAGCCCAGAGGTAAGGTGGTCAGACTCGGCTTCCTTCCCCGGAG
CTGAGAGGGAGGGGAACGTGGGGCAGATGCACAGGAAGAATTTCTGTGC
TCTGCCCAGTTGTCTGCTTTAGATAAATATGTCTGGGGGTTATGGGGAG
AAGATAAAAGTGCCTGTGGGACCACAGACTCTCGCTGTGGTGGAGCTGG
GCCCTCTTACCCTCCCAAGCCTCGCCCCTCATCCCATCCCTGGGGGCCA
GGGGTGAGGGCGGCAGGAACCTCAAGGCTCTGAGAAAGTGCGTGGTGTG
TGTTGCCATTTTGGTCTCTTCTCCGGCGCGCCCGGGATCCACGCGTCTG
AATTCGA

To assess the functional role of the synthetic polynucleotide, the inventors cloned it into the CEWP lentiviral plasmid, upstream of an eGFP domain (FIG. 2A). The LV produced using this lentiviral plasmid will be referred to as the CD4-GFP LV. Next, the inventors used the CD4-GFP LV to transduce multiple cell lines and primary T cells. High eGFP expression was only detected in cells that naturally express CD4, indicating that the synthetic promoter was specific to CD4+ cells (FIG. 2B, 2C). In addition, percentage of eGFP+ cells was comparable in transduced primary CD4+ and CD8+ T cells, even the intensity of expression was significantly higher in CD4+ T cells (FIG. 2D).

To investigate whether the synthetic polynucleotide mimics CD4 expression, the inventors compared the expression of the exogenous CD4-GFP with the expression of the endogenous CD4 in transduced primary T cells. To stimulate CD4-promoter mediated transcription, T cells were activated with αCD3/αCD28. Upon activation, CD4-GFP expression-regulated under the control of the synthetic promoter-mimicked the pattern of endogenous CD4 expression (FIG. 3C). Although the expression of GFP did not completely mimic the expression pattern of CD3, GFP expression did not increase after antigenic stimulation of T cells, which usually occurs with strong viral or eukaryotic promoters (FIG. 3B, 4).

Overall, these results indicate that the synthetic polynucleotide can be used for T cell specific transgene expression, and that it mimics the pattern of expression of the endogenous human CD4 after activation.

So, the first embodiment of the present invention refers to a polynucleotide comprising SEQ ID NO: 7 or having an identity of sequence with SEQ ID NO: 7 of at least 95%.

In a preferred embodiment, the polynucleotide comprises a promoter sequence consisting of SEQ ID NO: 7 or having an identity of sequence with SEQ ID NO: 7 of at least 95%.

In a more preferred embodiment, the polynucleotide comprises a promoter sequence consisting of SEQ ID NO: 7.

In a more preferred embodiment, the polynucleotide consists of SEQ ID NO: 7.

In a more preferred embodiment, the polynucleotide of the invention comprises or consists of SEQ ID NO: 7 and accessory sequences containing restriction sites, for instance SEQ ID NO: 8.

The second embodiment of the invention refers to a genetic construct comprising any of the polynucleotides described above.

In a preferred embodiment, the genetic construct comprises the any of the polynucleotides described above as promoter operatively linked to a nucleotide sequence encoding a protein to drive its expression.

In a more preferred embodiment, the protein is a chimeric antigen receptor (CAR).

The third embodiment of the invention refers to a lentiviral vector comprising any of the polynucleotides, or the genetic constructs described above.

The fourth embodiment of the invention refers to a T cell or T cell-derived cell comprising any of the polynucleotides, or genetic constructs described above, or any genetic material introduced using the lentiviral vector described above.

The fifth embodiment of the invention refers to a any of the polynucleotides, genetic constructs, lentiviral vector, T cell or T cell-derived cell described above for use in therapy, for instance in cancer therapy.

So, a preferred embodiment involves the polynucleotides, genetic construct, lentiviral vectors, T cell or T cell-derived cell for use in cancer therapy.

Another preferred embodiment involves the polynucleotides, genetic construct, lentiviral vectors, T cell or T cell-derived cell for use in human cancer therapy.

The sixth embodiment of the invention refers to the use of any of the polynucleotides described above as a promoter to drive gene expression in T cells or T cell-derived cells.

In a preferred embodiment, the polynucleotide drives the expression of a nucleotide sequence encoding a CAR.

The seventh embodiment of the invention refers to the use of any of the genetic constructs or lentiviral vector described above to express a nucleotide sequence encoding a CAR in T cells or T cell-derived cells.

The eighth embodiment of the invention refers to a method for generating cells that express a transgene in a manner that mimics the expression of the human CD4 gene, which comprises transducing cells with a genetic construct comprising any of the polynucleotides described above as promoter operatively linked to a nucleotide sequence encoding a protein, or with any of the genetic constructs or the lentiviral vector described above.

In a preferred embodiment, the method comprises transducing T-cells or T-cell derived cells with any of the genetic constructs or lentiviral vector described above.

A preferred embodiment involves a method for generating CAR T-cells which comprises transducing T-cells with a genetic construct comprising any of the polynucleotides described above as promoter operatively linked to a nucleotide sequence encoding a CAR, or with any of the genetic constructs or the lentiviral vector described above.

In a preferred embodiment, the cells of the above-mentioned embodiments are human cells.

The present invention also refers to a method for treating patients, for instance patients suffering from cancer, which comprises the administration of CAR T-cells generated using any of the methods described above.

The synthetic polynucleotide described above can be used to restrict the expression of the CAR transgene exclusively to T cells. This can improve the efficacy of CAR-T cell therapies by:
1. Blocking the expression of the CAR transgene in packaging cells during LV generation, thereby resulting in the generation of lentiviral particles that will be free of CARs.
2. If the T cells collected from the patient are contaminated with other cell types, by blocking the expression of the CAR transgene in case of LV-mediated transduction of contaminating cells.

The membrane of the retroviral vectors is derived from the packaging cells and contain different proteins derived from those producing cells. The level of incorporation is highly variable and depends on the type of retroviral vector, the protein and its concentration. In this direction, it has been described that CAR-expressing retroviral particles present the CAR on its surface, thereby facilitating its binding to the cognate antigen. In fact, malignant B cells are transduced at higher levels with LVs displaying anti-CD19 CARs compared to LVs displaying non-binding control constructs. This is referred to as transduction targeting.

Several groups are investigating the feasibility of direct inoculation of CAR-coding vectors into the patient as a cheaper and quicker alternative to ex vivo generation of CAR-T cells and posterior re-infusion. However, there are multiple safety concerns to solve before this strategy can be applied into patients. The present invention could accelerate this translation by providing a tight control of CAR expression only on T cells. This can also be combined with transduction targeting to avoid transduction of leukemic cells. Therefore, this invention would be a valuable alternative or complement to the receptor-targeted LVs (RT-LVs).

In addition to the described application to improved CAR-T cells platforms, other potential applications of the invention relate to any gene therapy strategies aiming to modify T cells ex vivo or in vivo. For example, new gene therapy strategies for immunodeficiencies and autoimmune diseases could be developed based on the specific expression of the desired gene only on T cells.

The polynucleotide can be used to achieve T-cell specific expression during pluripotent stem cell differentiation to investigate T cell development in vitro or in vivo.

The invention can be also of interest for production of retroviral vectors expressing highly toxic proteins, by blocking production its production on the packaging cells. This would improve the quality of particle production since the particles will be free of toxic proteins.

In the context of the present invention the following terms are defined:
The term “comprising” means including, but it is not limited to, whatever follows the word “comprising”. Thus, use of the term “comprising” indicates that the listed elements are required or mandatory, but that other elements are optional and may or may not be present.
By “consisting of” means including, and it is limited to, whatever follows the phrase “consisting of”. Thus, the phrase “consisting of” indicates that the listed elements are required or mandatory, and that no other elements may be present.

### DESCRIPTION OF THE FIGURES

FIG. 1. DNA sequence of CD4 promoter. A) Scheme of the full length synthetic CD4 promoter showing the positions of the different regions included, each one identified with a different color. B) Nucleotide sequence of the different regions of the synthetic CD4 promoter. C) Table indicating the sequences included in the synthetic CD4 promoter showing the sequence ID and exact locations in the GeneBank nucleotide sequence database. The chimeric promoter contains 6 sequences (from Seq_1 to Seq_6) obtained from different positions of the CD4 genomic locus. A human CD4 HS4 enhancer defined in (1) and (2) was added to the construct in the 5′ region, with the 3′ region more homologous to the murine enhancer. Adjacent-to-promoter sequences defined by EMBL nucleotide sequence database were also added. A CD4 minimal promoter defined in (3) and (4) was added, as well as a region of CD4 defined as promoter by Eukaryotic Promoter Database (EPD) (chr12: 6789491-6789550), which varies in the 5′ region with respect to the promoter region defined in (3). An enhancer region defined in (5) was incorporated into the 3′ region.

FIG. 2. GFP expression levels under the control of the CD4 synthetic promoter in 4 different cell types. A) Representation of lentiviral plasmids used to generate LVs. The CD4-GFP plasmid has an eGFP domain under the control of the synthetic CD4 promoter. The CEWP plasmid, used as control, has an eGFP domain under the control of the CMV promoter. B) CD4+++ cells: Primary T cells and Jurkat (an immortalized T cell line), CD4+: Namalwa, and non-T cells (293T and BxPC-3) were transduced with CD4-GFP or CEWP LVs and 5 days later analysed for eGFP expression. The different histograms show the expression levels of eGFP on the different cell lines transduced with each plasmid. NTD: non-transduced cells. C) Percentage (left) and intensity (right) of positive GFP+ cells in the different cell types analysed. Two-tailed T test. D) Percentage (left) and intensity (right) of eGFP+ cells among the CD4+ and CD8+ T cell subsets.

FIG. 3. Kinetics of CD4-driven polynucleotide in primary T cells. A) Work-flow of the kinetics experiments. Briefly, cells were activated, transduced with the CD4-GFP LVs and cultured for 10 days until resting. Then, cells were stimulated with TransAct and eGFP/CD3 expression was determined by FACS at the indicated times. B) Fold change between the percentage of positive cells (CD3+ or eGFP+) at different timepoints post stimulation, and those at 0 h. ANOVA-two tails test, Bonferroni post T. **, p<0.01; ***, p<0.001. C) Fold change between the expression of endogenous CD4 mRNA and exogenous CD4-GFP mRNA at different timepoints post stimulation, and those at 0 h.

FIG. 4. GFP kinetics in CD4/CD8 and CD45RA/CD62L T cells subsets. A) Fold change of GFP positive cells analysed in the CD4+ (left) and CD8+ (right) T cells subsets compared to the fold change CD3, both compared to 0 h. B) Fold change of GFP positive cells analysed in the CD45RA+CD62L+ (top left), CD45RA-CD62L+ (top right), CD45RA-CD62L-(bottom left) and CD45RA+CD62L− (bottom right) T cells subsets compared to the fold change CD3, both compared to 0 h. ANOVA-two tails test, Bonferroni post T. *, p<0.05, **, p<0.01; ***, p<0.001.

### DETAILED DESCRIPTION OF THE INVENTION

The present invention is illustrated by means of the Examples set below without the intention of limiting its scope of protection.

### Example 1. Material and Methods

### Example 1.1. Polynucleotide Design and Synthesis

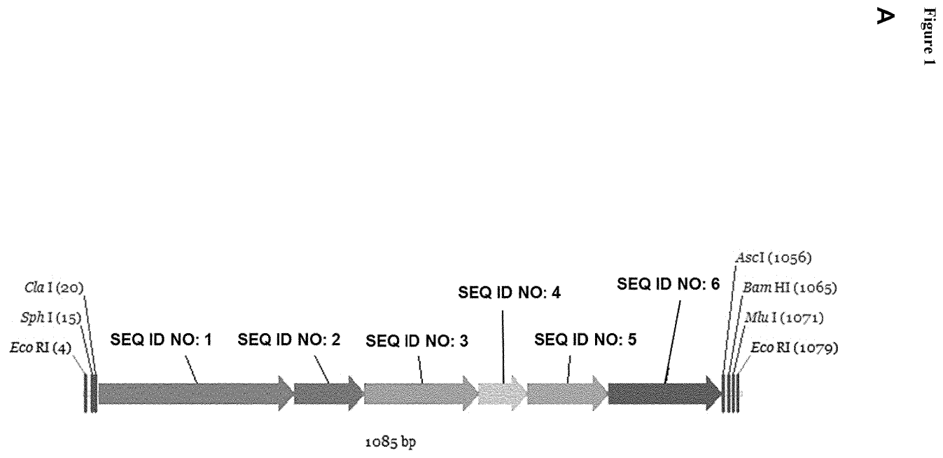

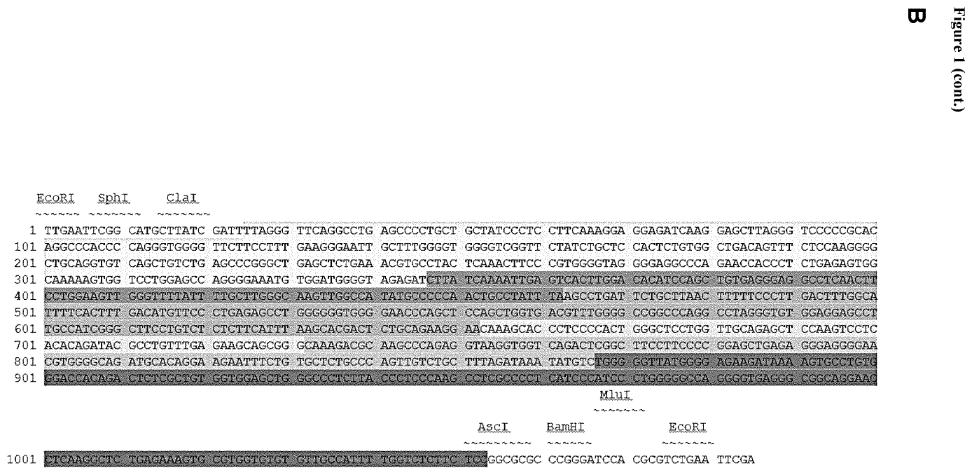

The synthetic polynucleotide (SEQ NO: 7, also referred to as the synthetic CD4 promoter) was designed based on CD4 endogenous locus. The inventors used different regions of the CD4 locus defined as important for regulation by different databases, genomic browsers or described as such in the literature. As shown in FIG. 1, SEQ ID NO:7 is composed of SEQ ID NO: 1, SEQ ID NO: 2, SEQ ID NO: 3, SEQ ID NO: 4, SEQ ID NO: 5 and SEQ ID NO: 6. SEQ ID NO: 8 consists of SEQ ID NO:7 and accessory sequences containing digestive restriction sites.

The polynucleotide was synthetized by GenScript (Genescript USA Inc. NY, USA) in a pUC57 vector, flanked by EcoRI/ClaI/SphI and BamHI/AscI/EcoRI restriction sites.

### Example 1.2. Lentiviral Plasmid Constructions

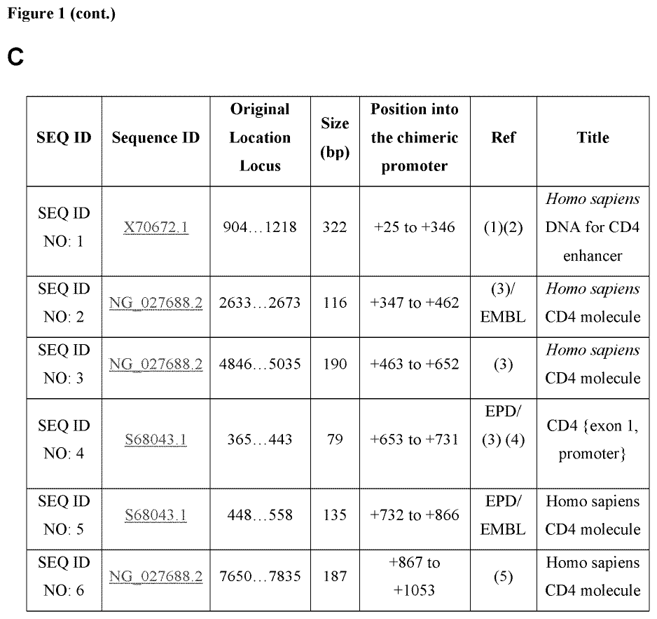

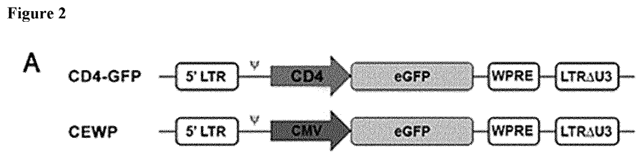

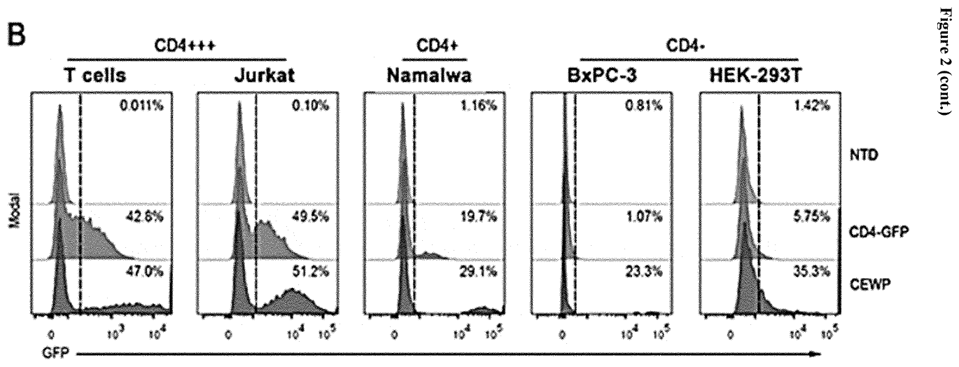

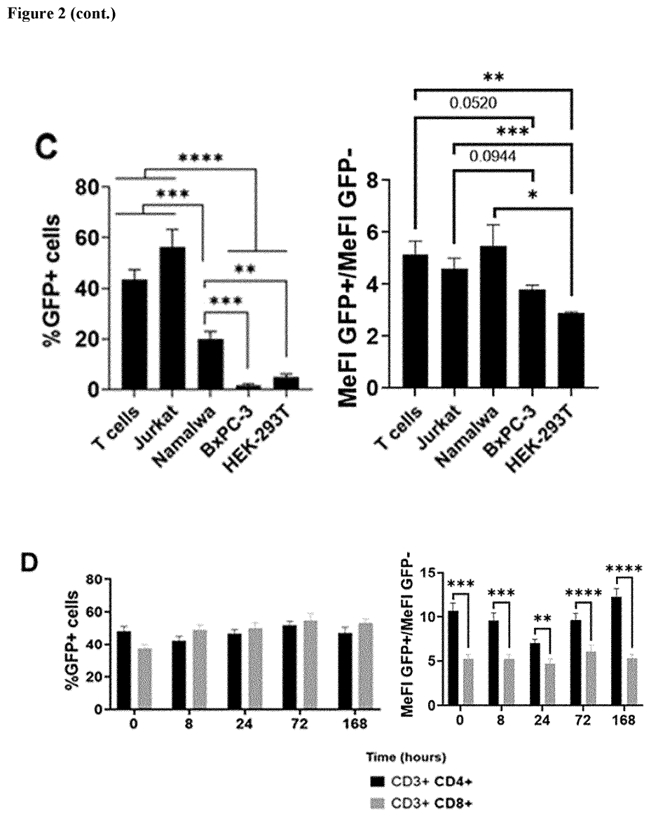

The synthetic polynucleotide was cloned into CEWP LV vector by using ClaI and BamHI with and standard techniques of molecular biology to generate what will be referred to as the CD4-GFP lentiviral plasmid. As shown in FIG. 2A, the CEWP lentiviral plasmid contains an eGFP domain, meaning that the resultant CD4-GFP lentiviral plasmid contains eGFP domain under the regulation of the synthetic CD4 promoter.

### Example 1.3. Cell Culture

HEK-293T (ATCC® CRL-11268) were cultured in DMEM (Biowest) supplemented with 10% FBS (Biowest) and maintained at 10% CO₂ and 37° C. Namalwa (ATCC® CRL-1432), Jurkat (ATCC® TIB-152), BxPC3 (ATCC® CRL-1687) cells were cultured in RPMI and MiaPaCa2 (ATCC® CRL-1420) in DMEM supplemented with 10% FBS and 1% P/S (Biowest) and maintained at 5% CO2 37° C. Primary T cells were isolated from the peripheral blood mononuclear cell (PBMC) section after Ficoll gradient (Lymphosep, Biowest) from peripheral blood and negative selected with the MACSexpresss Pan T cell isolation (Miltenyi Biotec). Isolated T cells were cultured in TexMACS medium (Miltenyi Biotec) supplemented with 20 UI/ml of IL-2 (Miltenyi) and 1% of P/S and maintained at 37° C. in a 5% CO₂ incubator. T cells were activated with TransAct (1:100, Miltenyi Biotec) during 48 h before lentiviral transduction following manufacturer's recommendations.

### Example 1.4. Generation of LVs

Using 6-well plates, 0.7-0.8×10⁶ HEK293T (90% of confluency) were transfected with the lentiviral plasmid of interest, pCMV_R8.91 and pMD2.G in proportion 3:2:1 using LipoD293 (SigmaGen Lab). DNA and LipoD were mixed in DMEM without serum and incubated for 10 min at room temperature before being added drop-by-drop to the cells. Media was exchanged 5 h post-transfection and supernatants were harvested at 48 h and 72 h after transfection and filtered through a 0.45 μm filter (Nalgene, Rochester, NY) and concentrated 30× using Amicon Ultra15 (100 KD, Milipore) filters and centrifugation at 1800 g 4° C. LVs were aliquoted and stored at −80° C.

Briefly, for the estimation of the efficient viral titer, 10⁵ cells were incubated with different volume of viral supernatant. Percentage of eGFP+ cells was determined by flow cytometry 72 h post-transduction and transducing units per ml (TU/ml) were estimated according to the formula:

$$Titer = \frac{100000 plated cells \times \% GFP + cells \times 1000}{micr oliters of LVs used}$$

The LVs produced upon transfection with the CD4-GFP and the CEWP lentiviral plasmids will be referred to as the CD4-GFP LV and the CEWP LV, respectively.

### Example 1.5. Transduction

Briefly, 10⁵ cells of suspension Jurkat and Namalwa cells were incubated with different volume of viral supernatants during 4.5 h post-spinoculation (30 min at 32° C. 800 g). In the case of adherent cells, 5×10⁴ cells of MiaPaCa2 and BxPC3 cells or 1×10¹ HEK-293T cells were plated with viral supernatants in a 24-well-plate and media was exchange after 5 h of incubation with LVs. For T cell transduction, 2×10⁵ activated T cells were incubated with LVs in s 96 well-plate and spinoculated at 800 g for 1 h at 32° C. After 5 h, cells were washed and plated at a density of 10⁶ c/ml. Cell transduction was evaluated by flow cytometry.

### Example 1.6. Analysis of Expression Kinetics in Primary T Cells

For T cells transduced with eGFP-LVs, 10⁵ T cells were stimulated with TransAct (Miltenyi Biotec) after 10 days of the initial activation for transduction. Fold change of eGFP expression was indicated as the ratio of percentage of positive population related to percentage of positive population at 0 h.

### Example 1.7. Statistical Analysis

Data are represented as mean±SEM. Two-tailed ANOVA, Bonferroni post-test has been used to compared kinetics as indicated in every figure caption.

### Example 2. Results

### Example 2.1. Construction of T-Specific Chimeric Promoters Based on Different Regulatory Regions of the CD4 Locus

To construct a T-cell specific promoter, the inventors used different regions of the CD4 locus defined as important for regulation by different databases, genomic browsers or described as such in the literature. The regions used are described in FIG. 1.

### Example 2.2. T-Cell Specific Expression of the CD4 Synthetic Promoter in a LV Background

LVs expressing eGFP under the regulation of the synthetic CD4 promoter and CEWP-LVs (as control of eGFP expression) were constructed (FIG. 2A). LVs were generated and used to transduce 293T and BxPC-3 cells as non-CD4-expressing cells, Namalwa (slightly positive for CD4) and Jurkat cells (an immortalized T cell line) and primary human T cells as CD4+++ cells (FIG. 2B, 2C). These results show that eGFP is only expressed in Jurkat, primary T cells and Namalwa, with almost undetectable levels of expression in 293T and BxPC-3. In primary T cells, the expression of eGFP was similar in CD4 and CD8 subsets in terms of percentage (FIG. 2D, left), but with an increased intensity in CD4+ population (FIG. 2D, right), suggesting that the promoter could be used to ensure CAR expression in CAR-T cells. Very low levels of eGFP expression were detected in 293T cells (5.75%), suggesting that the use of this promoter could prevent transgene expression in packaging cells during lentiviral generation. For instance, it could be used to prevent CAR expression on the surface of packaging cells.

### Example 2.3. TCR-Like Expression of the CD4 Polynucleotide

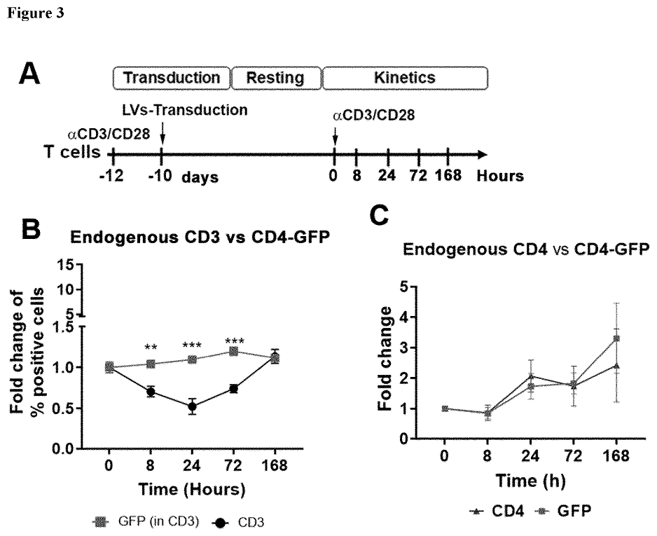

To analyse if the CD4 promoter-driven LV mimics the TCR expression pattern, the inventors transduced human T cells with CD4-LVs and analysed the kinetics of transgene expression at different times after T cell activation with αCD3/αCD28, as shown in FIG. 3A. They compared the effects of T cell activation on the percentage of CD3+ cells and eGFP+ cells at different timepoints after stimulation (FIG. 3B). As previously reported, CD3 expression in T cells decreased soon after stimulation, and basal levels were recovered after 3-7 days. However, such downregulation was not detected in GFP expression, which was driven by the CD4 polynucleotide. Instead, GFP expression levels remained unaltered upon stimulation, suggesting that the use of the CD4 polynucleotide could prevent non-desirable transgene overexpression (FIG. 3B). The inventors also compared the effects of T cell activation on endogenous CD4 and exogenous CD4-GFP mRNA levels in transduced T cells at different timepoints post stimulation. This revealed that both levels followed similar dynamics, suggesting that expression under the synthetic CD4 promoter follows a CD4-like behaviour (FIG. 3C).

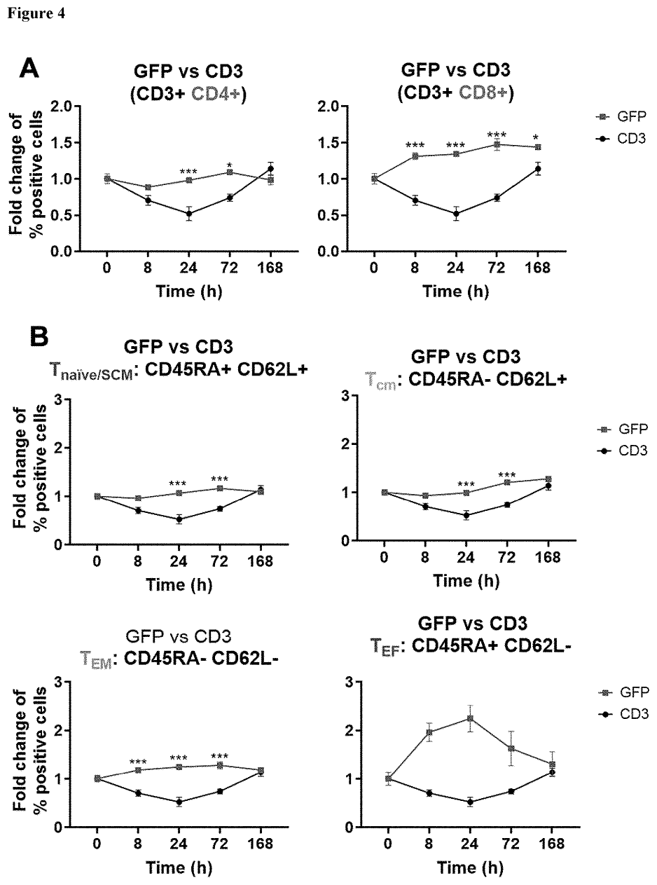

In addition, the inventors also determined transgene expression in CD4+ CD3+ and CD8+ CD3+ T cells subsets at different stages of T cells differentiation (FIG. 4). In CD4+ cells, GFP expression slightly mimicked CD3 expression in CD4+ cells, while it showed a slight increment in CD8+ cells (FIG. 4A). The highest consistency between the GFP and the CD3 expression profiles was detected in naive/stem memory T cells and central memory T cells, while an increment in GFP compared to CD3 expression was observed in effector T cells. In the context of the generation of CAR-T cells from donors, this analysis evidences the importance of evaluating T cell phenotypes, in order to select less differentiated T cell populations for the generation of CAR-T cells.

1. A polynucleotide comprising SEQ ID NO: 7 or having an identity of sequence with SEQ ID NO: 7 of at least 95%.

2. Polynucleotide, according to claim 1, comprising a promoter sequence consisting of SEQ ID NO: 7 or having an identity of sequence with SEQ ID NO: 7 of at least 95%.

3. Polynucleotide, according to any of the claim 1 or 2, comprising a promoter sequence consisting of SEQ ID NO: 7.

4. Polynucleotide, according to any of the claim 1 or 2, consisting of SEQ ID NO: 7.

5. A genetic construct comprising the polynucleotide according to any of the claims 1 to 4.

6. Genetic construct, according to claim 5, comprising the polynucleotide according to any of the claims 1 to 4 as promoter operatively linked to a nucleotide sequence encoding a protein to drive its expression.

7. Genetic construct, according to claim 6, wherein the protein is a chimeric antigen receptor (CAR).

8. Lentiviral vector comprising the polynucleotide of any of the claims 1 to 4, or the genetic construct of any of the claims 5 to 7.

9. T cell or T cell-derived cell comprising the polynucleotide according to any of the claims 1 to 4, a genetic construct according to any of the claims 5 to 7, or any genetic material introduced using the lentiviral vector of claim 8.

10. The polynucleotide according to any of the claims 1 to 4, the genetic construct according to any of the claims 5 to 7, the lentiviral vector according to claim 8, or the T cells or T cell-derived cell according to claim 9, for use in therapy.

11. Use of the polynucleotide according to any of the claims 1 to 4 as a promoter to drive gene expression in T cells or T cell-derived cell.

12. Use, according to claim 11 to drive the expression of a nucleotide sequence encoding a CAR.

13. Use of the genetic construct, according to any of the claims 5 to 7, or the lentiviral vector according to claim 8, to express a nucleotide sequence encoding a CAR in T cells or T cell-derived cells.

14. Method for generating cells that express a transgene in a manner that mimics the expression of the human CD4 gene, which comprises transducing cells with a genetic construct comprising the polynucleotide according to any of the claims 1 to 4 as promoter operatively linked to a nucleotide sequence encoding a protein, or with a genetic construct according to any of the claims 5 to 7, or with the lentiviral vector according to claim 8.

15. Method, according to claim 14, for generating CAR T-cells which comprises transducing T-cells with a genetic construct comprising the polynucleotide according to any of the claims 1 to 4 as promoter operatively linked to a nucleotide sequence encoding a CAR, or with a genetic construct according to any of the claims 5 to 7, or with the lentiviral vector according to claim 8.
